(19) United States
(12) Plant Patent Application Publication
(10) Pub. No.: US 2004/0139515 P1
(43) Pub. Date: Jul. 15, 2004

(54) CHRYSANTHEMUM PLANT NAMED 'CHASEIZE'

(76) Inventor: Jean-Pierre Challet, Nuaille (FR)

Correspondence Address: BURNS DOANE SWECKER & MATHIS L L P, POST OFFICE BOX 1404, ALEXANDRIA, VA, 22313-1404, US

(21) Appl. No.: 10/342,316

(22) Filed: Jan. 15, 2003

(51) Int. Cl. A01H 5/00

(52) U.S. Cl. PLT/263

(57) ABSTRACT

A new and distinct Charm Chrysanthemum cultivar is provided that is the result of a controlled breeding program. Attractive small daisy-like blossoms are formed in profusion in clusters having green disc florets that change to yellow with maturity and two rows of pink ray florets that change to light pink with maturity. The growth habit is bushy, dense, and well-branched with short internodes and yields a generally uniform and spherical overall plant. The foliage is small and gray-green in coloration with near white nervures, and blends nicely with the blossom coloration. The response time is approximately eight weeks. The natural flowering time is late-October. When mature the blossoms emit pollen that is orange in coloration. The plant grows well in pots.

### BOTANICAL/COMMERCIAL CLASSIFICATION

Ajania pacificum/Decorative Pot Mum

### VARIETAL DENOMINATION

cv. ‘Chaseize’

### SUMMARY OF THE INVENTION

The present invention comprises a new and distinct cultivar of Chrysanthemum, botanically known as Ajania pacificum, and hereafter is referred to by the cultivar name ‘Chaseize’.

The new cultivar of the present invention was created at Nuaillé, France during the course of a controlled breeding program. The seed parent (i.e., the female parent) was an unnamed plant of the species, and the pollen parent (i.e., the male parent) was designated ‘92/27/3’. Neither parent was patented in the United States. The seeds resulting from the cross were sown and small plants were obtained which were physically and biologically different from each other. Selective study resulted in the identification of a single plant of the new cultivar of the present invention.

It was found that the new Charm Chrysanthemum cultivar of the present invention displays:

(a) a bushy, dense, well-branched, and generally spherical growth habit with short internodes,

(b) profusely forms attractive small daisy-like blossoms having green disc florets that change to yellow with maturity, and two rows of pink ray florets that change to light pink with maturity,

(c) forms decorative small gray-green leaves with near white nervures,

(d) forms orange-colored pollen when the blossoms are mature, and

(e) an ability to grow well in pots.

The new cultivar can be grown singly or in clumps in pots. It also can be grown in the landscape. The yellow and pink blossoms blend nicely with the gray-green foliage. The plant is self-branching and the internodes are short. Pinching is helpful to further enhance branching and produces a large number of shoots; however, such pinching is not necessary since the plant already is inherently well branched. The natural flowering time is late-October.

Asexual reproduction of the new cultivar by the use of cuttings as performed at Nuaillé, France, in a controlled environment has demonstrated that the characteristics of the new cultivar are firmly fixed and are retained through successive generations of asexual propagation.

‘Chaseize’ has not been observed under all possible environmental conditions to date. Accordingly, it is possible that the phenotype may vary somewhat with variations in the environment, such as temperature, light, day length, contact with pesticides and/or subjection to grown regulation treatments.

### BRIEF DESCRIPTION OF THE PHOTOGRAPH

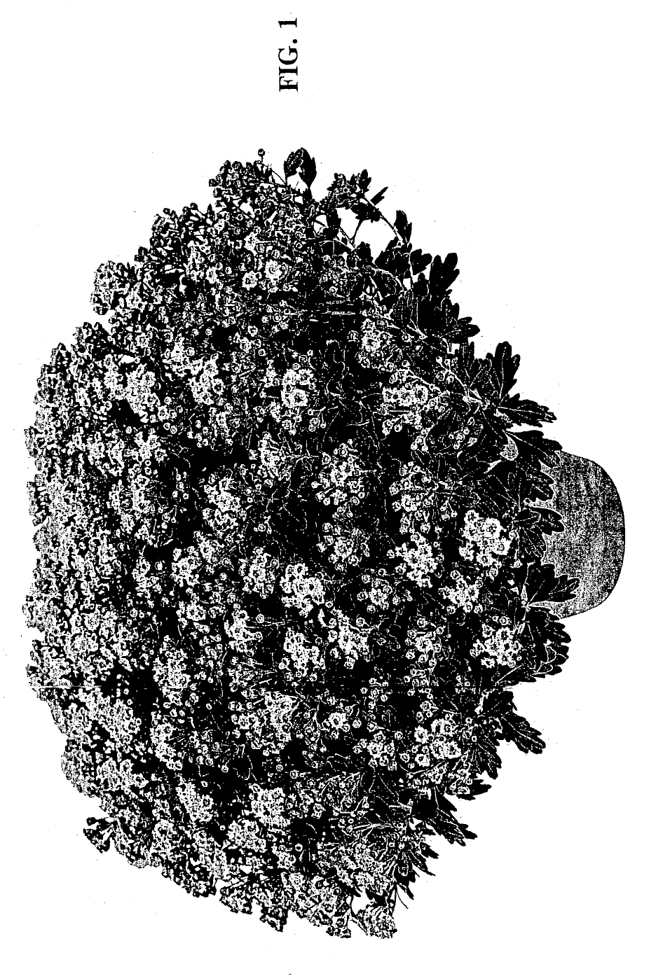

The accompanying FIG. 1 depicts a typical eighteen week-old plant of ‘Chaseize’ cultivar while growing in a pot in a greenhouse at Nuaillé, France. The attractive compact spherical growth habit, with a substantially homogenous coverage of yellow and pink blossoms, and gray-green foliage with near white nervures are illustrated.

### DETAILED DESCRIPTION

The plants described were grown in pots in a greenhouse at Nuaillé, France. No growth regulation was used. The growing conditions approximate those commonly utilized for the commercial production of decorative pot mums.

General appearance: Bushy and dense with a generally spherical growth habit. The internodes are short and commonly measure approximately 10 to 25 mm on average. When a cutting is placed during week 23 in a four-liter pot, the resulting plant commonly will display a height of approximately 30 to 32 cm on average and a width of approximately 50 to 55 cm on average at the end of flowering during late October.

Foliage: Attractive gray-green. The main exposed nervures are near white in coloration.

Flowers: The attractive small daisy-like blossoms are formed in clusters. The flower coverage is substantially homogeneous across the plant. Accordingly, the flowers are so closely grouped as to substantially cover the circumference of the dome-shaped plant. The disc florets initially are green and change to yellow with maturity. The ray florets are present in two rows and are pink and change to light pink with maturity. The flower diameter when mature commonly ranges from approximately 10 to 15 mm. Orange pollen commonly is displayed in the mature blossoms.

Flower time: The natural flowering time is late-October.

Response time: The time between the beginning of the short day period and the flowering date at normal temperature and light intensity is approximately eight weeks.

Usage: Decorative pot mum.

I claim:

1. A new and distinct cultivar of Charm Chrysanthemum plant that displays:
(a) a bushy, dense, well-branched, and generally spherical growth habit with short internodes,
(b) profusely forms attractive small daisy-like blossoms having green disc florets that change to yellow with maturity, and two rows of pink ray florets that change to light pink with maturity,
(c) forms decorative small gray-green leaves with near white nervures,
(d) forms orange-colored pollen when the blossoms are mature, and
(e) an ability to grow well in pots;
substantially as illustrated and described.
